(19) United States
(12) Patent Application Publication
(10) Pub. No.: US 2026/0083904 A1
(43) Pub. Date: Mar. 26, 2026

(54) CARTRIDGE ASSEMBLY AND HUB FOR INFUSION CATHETER AND CGM SENSOR INSERTION AND INTRODUCER NEEDLE REMOVAL

(71) Applicant: AITA BIO INC., New York, NY (US)

(72) Inventors: Dilan Casanovas Mack, New York, NY (US); Russell Cole, River Vale, NJ (US)

(21) Appl. No.: 19/106,604

(22) PCT Filed: Sep. 16, 2023
(86) PCT No.: PCT/US23/32968

§ 371 Date: Feb. 25, 2025

Related U.S. Application Data
(60) Provisional application No. 63/415,421, filed on Oct. 12, 2022.

(51) Int. Cl. A61M 5/142; A61M 5/14; A61M 5/158

(52) U.S. Cl. CPC A61M 5/14248; A61M 5/1413; A61M 5/158; A61M 2005/14268; A61M 2005/1585; A61M 2202/04; A61M 2205/33; A61M 2210/04

(57) ABSTRACT

An infusion system comprising: a device for delivering fluid to a tissue of the user; a hub engaging the device for delivering fluid, the hub including an introducer needle for introducing an infusion catheter or an analyte sensor into the tissue of the user, the hub configured to be removable by the user; and an activation mechanism for moving the hub toward the user to thereby cause the introducer needle to be inserted into the tissue of the user.

### CROSS REFERENCE TO RELATED APPLICATIONS

This application claims priority to U.S. provisional application No. 63/415,421, filed Oct. 12, 2022, entitled “Cartridge Assembly With Infusion Catheter and CGM Sensor For Simultaneous Insertion” which is incorporated by reference herein.

### FIELD OF INVENTION

The present invention relates to a cartridge assembly and hub for infusion catheter and CGM sensor insertion and introducer needle removal.

### BACKGROUND OF THE INVENTION

Insulin pumps help people with diabetes to conveniently manage their blood sugar. These devices deliver insulin at specific times. Insulin patch pumps or pods are one type of insulin pump. The pods are wearable devices that adhere to the skin of a user using an adhesive patch. The pods deliver insulin from a chamber and internal cannula based on CGM sensor readings that are acquired by a separate device. Further, these devices are both bulky and cumbersome to apply and wear. Consequently, the pods protrude and catch on clothing in warmer weather and draw unwanted attention due to size. In some case, the pods cases dislodge when colliding with door frames and chairs during user movement.

It would be advantageous to provide improvements to insulin pumps and diabetes management described above.

### SUMMARY OF THE INVENTION

A cartridge assembly and hub for infusion catheter and CGM sensor insertion and introducer needle removal are disclosed.

In accordance with an embodiment of the present disclosure, an infusion system comprising: a device for delivering fluid to a tissue of a user; a hub engaging the device for delivering fluid, the hub including an introducer needle for introducing an infusion catheter or an analyte sensor into the tissue of the user, the hub configured to be removable by the user; and an activation mechanism for moving the hub toward the user to thereby cause the introducer needle to be inserted into the tissue of the user.

In accordance with another embodiment of the present disclosure, an infusion system comprising: a device for delivering fluid to a user, the device including a pump for pumping the fluid into a tissue of the user; a cartridge assembly including an infusion catheter and an analyte sensor to be inserted into the tissue of the user, the needle cartridge assembly configured to move from (1) a first position, wherein infusion catheter and analyte sensor are in a retracted position above the tissue of the user to (2) a second position, wherein the infusion catheter and analyte sensor are in a deployed position inserted into the tissue of the user; and a hub configured to engage the cartridge assembly and to be removed from the cartridge assembly, the hub including first and second introducer needles for introducing the infusion catheter and the analyte sensor, respectively into the tissue of the user.

In accordance with an embodiment of the present disclosure, an infusion system comprising: a device for delivering insulin to a user, the device including a pump for pumping the insulin into a subcutaneous tissue layer of the user; a cartridge assembly including an infusion catheter and an analyte sensor to be inserted into the subcutaneous tissue layer of the user, the needle cartridge assembly configured to move from (1) a telescoping position above the top surface of device, wherein the introducer needle is in a retracted position within the device for delivering insulin to the user and (2) an advanced position within the device, wherein the introducer needle is in a deployed position inserted into the subcutaneous tissue layer of the user along with the catheter and the analyte sensor; a hub configured to be removed from the cartridge assembly, the hub including first and second introducer needles for introducing the infusion catheter and the analyte sensor, respectively into the subcutaneous tissue layer of the user; and a detachable activation mechanism for causing the cartridge assembly to move from (1) the telescoping position to (2) the advanced position within the device.

### BRIEF DESCRIPTION OF DRAWINGS

FIGS. 1 and 2 depict bottom perspective views of an infusion system for infusing insulin into a user in pre-activation and post activation configurations, respectively.

FIG. 3 depicts a view of a cartridge assembly of FIG. 1.

FIG. 4 depicts a view of the cartridge assembly of FIGS. 1 and 2 with introducer needles removed.

FIG. 5 depicts a side view of the cartridge assembly fluidically and electrically connected to the device (or pod) for delivering insulin to the user.

FIG. 6 depicts an exploded view of the cartridge assembly components.

FIG. 7 depicts a view of the portion of the device (or pod) for delivering insulin to the user exposing inner components.

FIG. 8 depicts a view of another example cartridge assembly.

FIG. 9 depicts an exploded view of the cartridge assembly shown in FIG. 8.

FIG. 10 depicts a side view of the cartridge assembly installed within device (or pod) for delivering insulin to the user but before it is assembled into detachable activation mechanism.

FIG. 11A depicts a longitudinal cross sectional view of the infusion system in which device (or pod) for delivering insulin to the user is in a pre-activation configuration that is assembled into the detachable activation mechanism.

FIG. 11B depicts a cross-sectional view of the infusion system in FIG. 11A along line 11B-11B.

FIG. 11C depicts a cross-sectional view of the infusion system in FIG. 11A along line 11C-11C.

FIG. 12 depicts a transparent view of the infusion system in FIG. 1 wherein the device (or pod) for delivering insulin to the user is in an post-activation configuration wherein the needles are driven into user tissue.

FIGS. 13 and 14 depict views of the device (or pod) for delivering insulin to the user where after activation, the user has removed the detachable activation mechanism.

FIGS. 15 and 16 depict views of the device (or pod) for delivering insulin to the user wherein the introducer needles have been removed using an insertion needle hub.

FIG. 17 depicts a block diagram of example components of the device for delivering insulin and needle cartridge assembly of the infusion system.

### DETAILED DESCRIPTION OF THE INVENTION

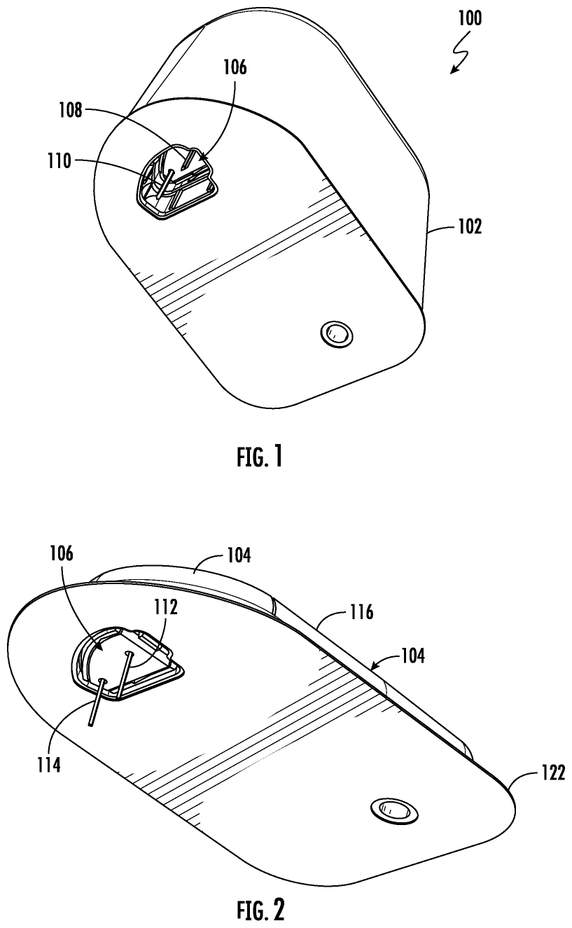

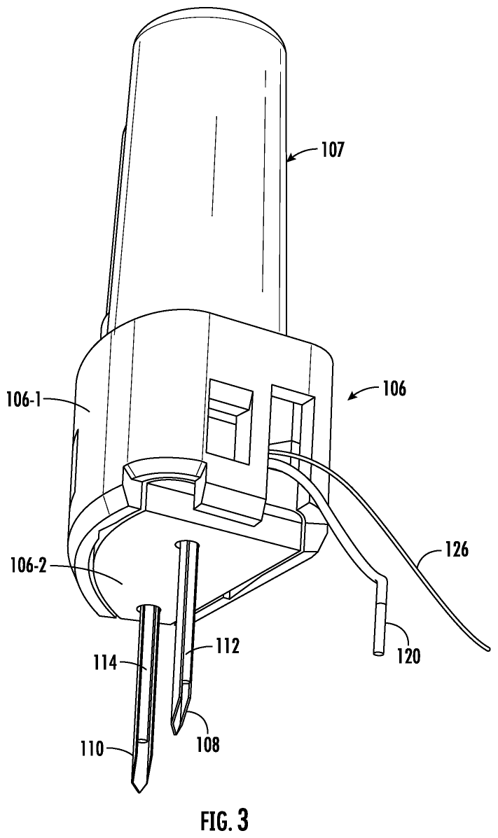

FIGS. 1 and 2 depict bottom perspective views of infusion system 100 for infusing insulin (or other fluid medication) into a user (patient) in pre-activation and post activation configurations, respectively. Infusion system 100 comprises a detachable activation mechanism 102 and device 104 (or pod) for delivering insulin to the user. However, those skilled in the art know that device 104 may deliver fluidic medication to the user other than insulin. Example medication may include small molecule pharmaceutical solutions, large molecule or protein drug solutions, saline solutions, blood or other fluids known to those skilled in the art. Insulin is an example of fluid that is described in this application.

As described in more detail below, device 104 is a wearable apparatus, system or pod for diabetes management in which continuous glucose monitoring (CGM), insulin delivery and control functionality are provided to ensure insulin is delivered at very precise rates and has the capability of detecting occlusions in real time. Detachable activation mechanism 102 compartment is outside of the hermetically sealed compartment of device 102 that contains a pump and electronics so water ingress can occur without affecting device performance. This is described in more detail below.

In brief, device 104 is preassembled in a single use detachable activation mechanism 102. Upon activation, detachable activation mechanism 102 releases a spring which drives, via a hub and cartridge assembly, introducer needles 108 and 110 coupled with infusion catheter 112 and continuous glucose monitoring (CGM) sensor 114 (as one form of an analyte sensor) into the skin. Detachable activation mechanism 102 passively detaches upon activation. The springs, activation components and device 102 release components are removed with mechanism 102 which is disposed of. As mentioned CGM sensor is an example analyte sensor that may be used. However, other analyte sensors may be used in device 104 for delivering insulin or other fluid.

In more detail, detachable activation mechanism 102 includes detachable mechanism housing 102-2, activation button 102-4, insertion spring 102-6, activation beam 102-8, inner housing 102-10, left release arm 102-12, right release arm 102-14 opposing left release arm 102-12, release spring 102-16, release catch 102-18, release catch screw 120, insertion rod 102-22 and a number of screws for assembling these components (shown but not numbered). Right release arm 102-14 is identical to release arm 102-12. Left and right release arms 102-12,102-14 includes retention flange 102-12a, 102-14a, respectively that fit within groove 124 that is part of and extends around bottom of device 104. (In this embodiment, CGM sensor 114 is employed, but those skilled in the art know that other analyte sensors may be used to achieved desired results as described herein.) Introducer needles 108,110 remain attached to the device 104 (pod) until removed by the user. In a bit more detail, device 104 is applied by opening a sterile packaging, filling the reservoir with insulin, priming the fluid path, removing the adhesive backing, sticking infusion system 100 to the desired body location, pushing a button assembly, removing and disposing the detachable activation mechanism 102. Removing detachable activation mechanism 102 components that are necessary for device 104 activation and needle insertion, but not required for actual infusion or sensing offers the benefit of a smaller, lower profile, more comfortable and discrete wearable device 104 for the user. This is described in more detail below.

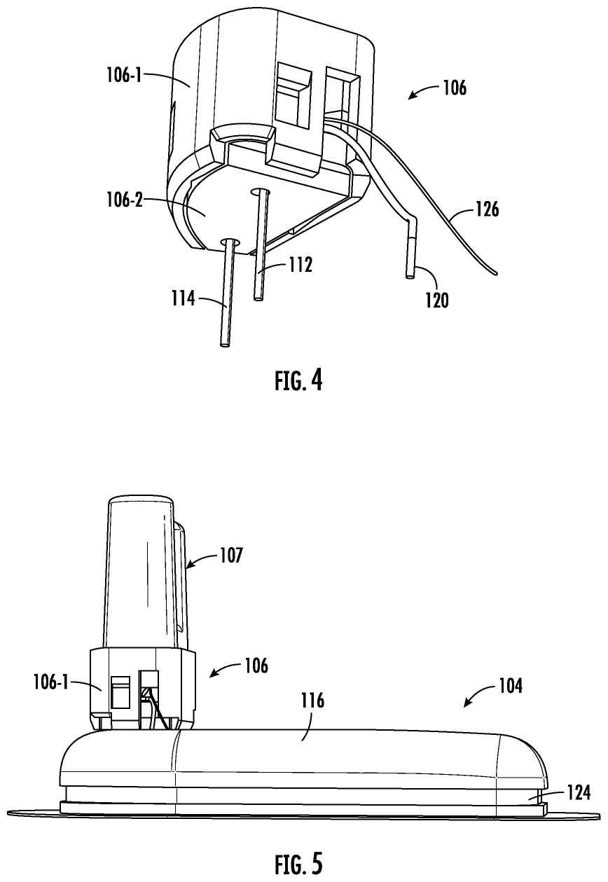

Detachable activation mechanism 102 is configured to insert infusion catheter 112 and CGM sensor 114 of device 104, simultaneously, at an insertion site normal to a user's skin or at other desired angles known to those skilled in the art. FIGS. 2 and 5 clearly show device 104. In the embodiment described herein, specifically, detachable activation mechanism 102 and needle cartridge assembly 106 are described with respect to inserting two separate introducer needles 108,110 as described below to insert an infusion catheter 112 and CGM sensor 114 simultaneously. Introducer needles 108,110 are preferably constructed of steel, but it may be any rigid material known to those skilled in the art. Introducer needles 108,110 (and thus infusion catheter 112 and CGM sensor) are separated so as to avoid insulin interference with sensor readings.

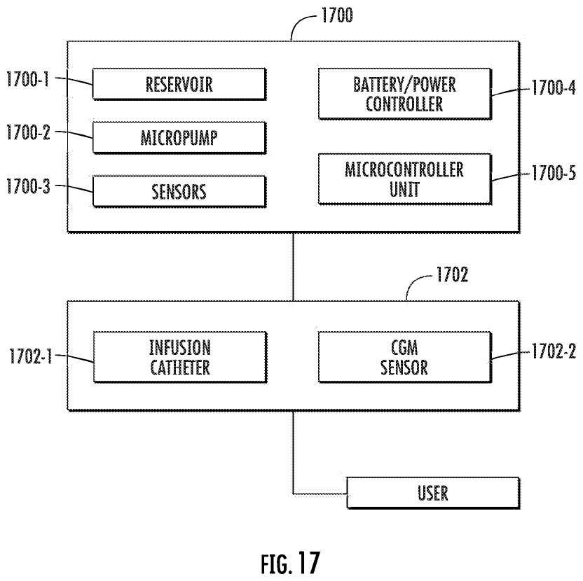

Device 104 incorporates, among other elements (as described below), a micropump (micropump 1700-2 in FIG. 17) as known to those skilled in the art that can be used for pumping fluid, valves used for regulating flow, actuators used for moving or controlling the micropump and valves and/or sensors used for sensing pressure and/or flow. The micropump may be used to infuse the insulin or other fluidic medication to the user (patient). Medication may include small molecule pharmaceutical solutions, large molecule or protein drug solutions, saline solutions, blood or other fluids known to those skilled in the art. Insulin is an example of fluid that is described in this application. However, micropump may be used in other environments known to those skilled in the art. In addition, device 104 may employ other kinds of pumps (other than micropumps).

Device 104 also includes reservoir (reservoir 1700-1 in FIG. 17), a microcontroller unit (MCU) (MCU 1700-5 in FIG. 17), infusion catheter 112 and CGM sensor 114 and a battery and power controller (Controller 1700-4 in FIG. 17). The reservoir is configured to receive and store insulin for its delivery over a course of about three days, or as needed. However, the reservoir size may be configured for storing any quantity of fluid as required. The micropump fluidly communicates with reservoir to enable infusion as needed. CGM, as known to those skilled in the art, tracks patient glucose levels and permits those levels to be used in algorithms that control flow rate. MCU controls the operation of the micropump to deliver insulin through the insulin catheter 112 from the reservoir at specific doses, i.e., flow rates over specified time intervals, based on CGM data converted to desired flow rate via control algorithms. The battery and power controller controls the power to the MCU and the micropump to enable those components to function properly as known to those skilled in the art. The CGM is powered by battery and the power controller through the MCU.

Infusion system 100 further incorporates cartridge assembly 106 as shown in FIGS. 1-16 along with introducer needle hub 107 extending from cartridge assembly 106. Cartridge assembly 106 is configured to fit within a cartridge (insertion) compartment opening 109 in top housing 116 (described below) and a channel extending through top housing 116 of device 104. The cartridge (insertion) compartment opening 109 in the top housing 116 enables access for the cartridge assembly 106 so it can drive introducer needles 108,110 into the subcutaneous tissue of a user (also may be referred to as a subcutaneous tissue layer herein). Introducer needle hub 107 is configured to be removable and function to withdraw introducer needles 108,110. By utilizing an introducer needle hub in this way, device 104 avoids storing or holding the needles during user operation. As a result, the size and weight of device 104 are drastically reduced, and more particularly the profile height of the device 104 (pod) is significantly reduced. This has serious benefits. Device 104 is discrete and more comfortable to apply and use. Further, the low profile device avoids issues with accidental dislodgement. This is described in more detail below.

Upon needle(s) insertion, opening 109 is closed off, so the housing provides some sealing properties and creates a continuous surface as shown in FIG. 2 for example. Detachable activation mechanism 102 compartment is outside of the hermetically sealed compartment that contains the pump and electronics of device 104 for delivering insulin so water ingress can occur without affecting device 104 performance. The amount of water ingress is limited by minimizing the volume of empty space in order to reduce the wetting nuisance that can occur after a user's swim or shower.

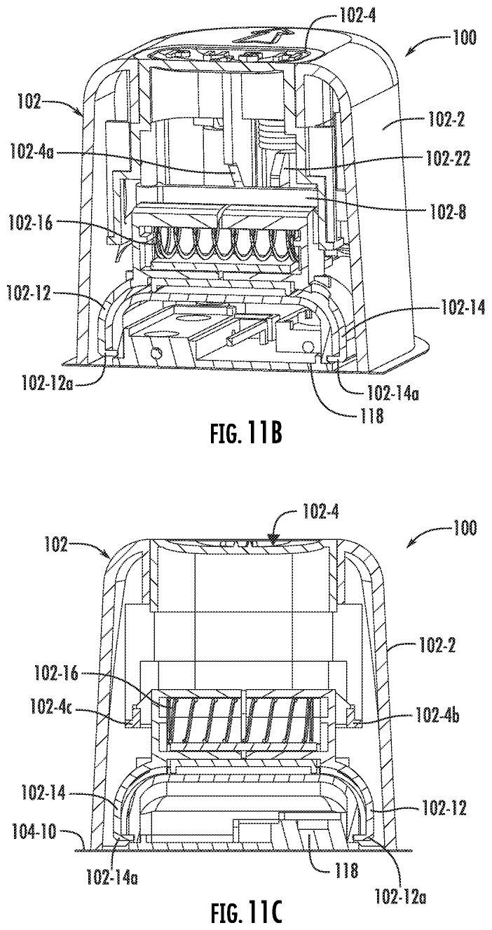

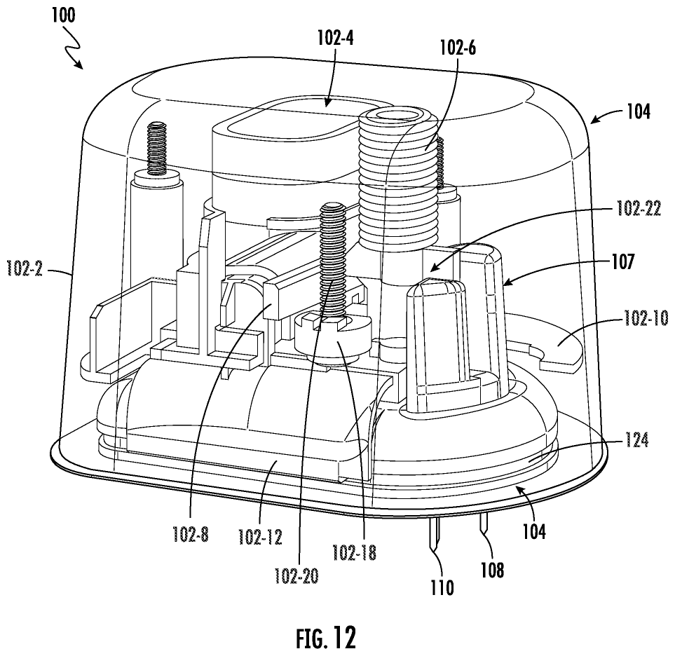

In brief, needle cartridge assembly 106 is configured to move from (1) a telescoping position above the top surface 116 of device 104 as shown in FIG. 11A for example, wherein infusion catheter 112 and CGM sensor 114 are in a retracted position to (2) an advanced position within device 104, as shown in FIG. 12 for example, wherein the infusion catheter 112 and CGM sensor 114 are in a deployed position embedded into the subcutaneous layer of the user's tissue (after insertion). While this is described in more detail below, insulin infusion catheter 112 and CGM sensor 114 are simultaneously inserted into the subcutaneous tissue layer for wearable insulin infusion delivery device 104 that includes a continuous glucose monitor (CGM).

Device 104 can be a closed loop or partially closed loop wearable insulin delivery device driven by blood glucose sensing feedback. Integrating these two capabilities into a single wearable device provides a more discreet and convenient means of optimizing blood glucose levels compared to the currently available commercial options. Combining insertion for CGM and infusion into a shared mechanism also has the advantages of reducing the number of insertion events users must endure. In the embodiments described herein as described above, introducer needles 108, 110 are separated accordingly but configured to simultaneously insert infusion catheter 112 and CGM sensor 114.

Device 104 for delivering insulin includes top housing 116, baseplate 118, introducer needles 108,110, infusion catheter 112 and CGM sensor 114, insertion mechanism tubing 120 and adhesive patch 122. Device 104 includes groove 124 along the lower periphery thereof that is defined by housing 116 and baseplate 118 in an assembled configuration. Groove 124 is used to receive flanges of the components of detachable activation mechanism 102 as described in more detail below.

In the embodiments described herein, infusion catheter 112 is preferably a PEEK infusion catheter or other materials with suitable biocompatibility, strength, flexibility and insulin compatibility, connected directly to the insulin fluid path that is inserted with introducer needle 108 that is preferably U-shaped. Other catheter materials and insertion designs are possible with mechanism 102 including an untipped stainless-steel catheter inserted with a U-shaped introducer needle and a standard Teflon catheter inserted with a hypodermic needle. Introducer needle 110 is also preferably U-shaped but it may be any shape as known to those skilled in the art.

FIG. 3 depicts cartridge assembly 106 that includes cartridge 106-1 and cartridge cover 106-2, also known as base insert 106-2, along with infusion catheter 112 and CGM sensor 114 secured within and extending from base insert 106-2. Cartridge assembly 106 is connected to an infusion fluid path and CGM electronics, as described below, and assembled into device 104 before being assembled into detachable activation mechanism 102. These are the components driven downward by the activation mechanism 102 to insert infusion catheter 112 and CGM sensor 114. Infusion catheter tubing 120 extends from infusion catheter 112 through a window in cartridge assembly 106 as shown. CGM connection wires 126 is connected to CGM sensor 114 and also extend through the same window as shown.

FIG. 4 depicts cartridge assembly 106 with introducer needles 108,110 removed to illustrate the configuration after insertion and during use. FIG. 5 depicts cartridge assembly 106 fluidically and electrically connected to the device 104. This is the configuration of device 104 before it is assembled into detachable activation mechanism 102. In this configuration, introducer needles 108,110 are coupled with infusion catheter 112 and CGM sensor 114 and cartridge subassembly 106 is in the upward pre-activation position (subcutaneous). In one embodiment, a PEEK infusion catheter is employed and the advantage of a PEEK infusion catheter is that it can connect directly to the insulin fluid path septum without the need for additional intermediate fluidic sealing components.

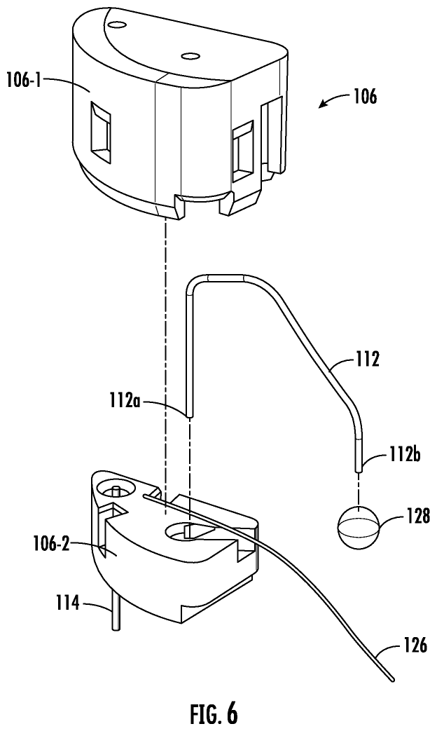

FIG. 6 depicts an exploded view of cartridge assembly 106 that may be installed in device 104 in pre-activation configuration. Infusion catheter 112 has patient end 112a and septum connecting end 112b of infusion catheter 112. Several components are shown including fluid path ball septum 128.

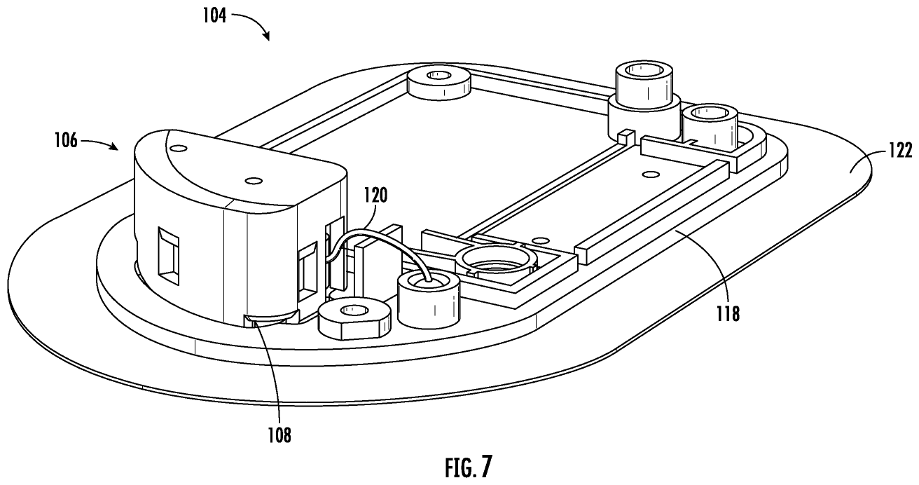

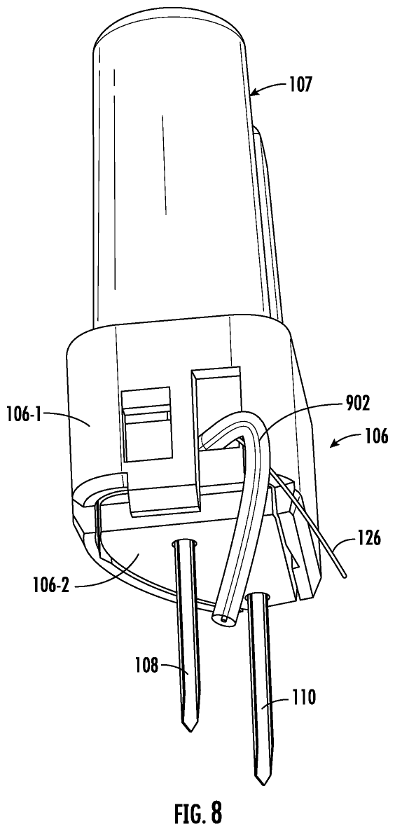

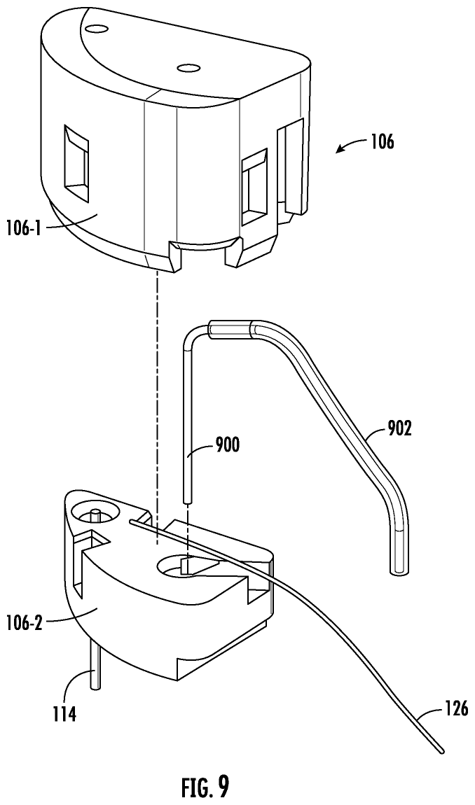

FIG. 7 depicts a portion of device 104 exposing inner components. Infusion catheter connection to the fluid path port in baseplate 118 is shown. Alternately, infusion catheter 900 can be comprised of an untipped (dull) stainless steel hypodermic tubing connected to the fluid path with flexible plastic tubing 902 as shown in FIG. 8. (The infusion catheter and CGM sensor are not shown in FIG. 8.) The stainless-steel tubing 900 would also be inserted by a U-shaped introducer needle 108. FIG. 9 depicts an exploded view of cartridge assembly 106 shown in FIG. 8 with CGM connection wires 126 but without hub 107.

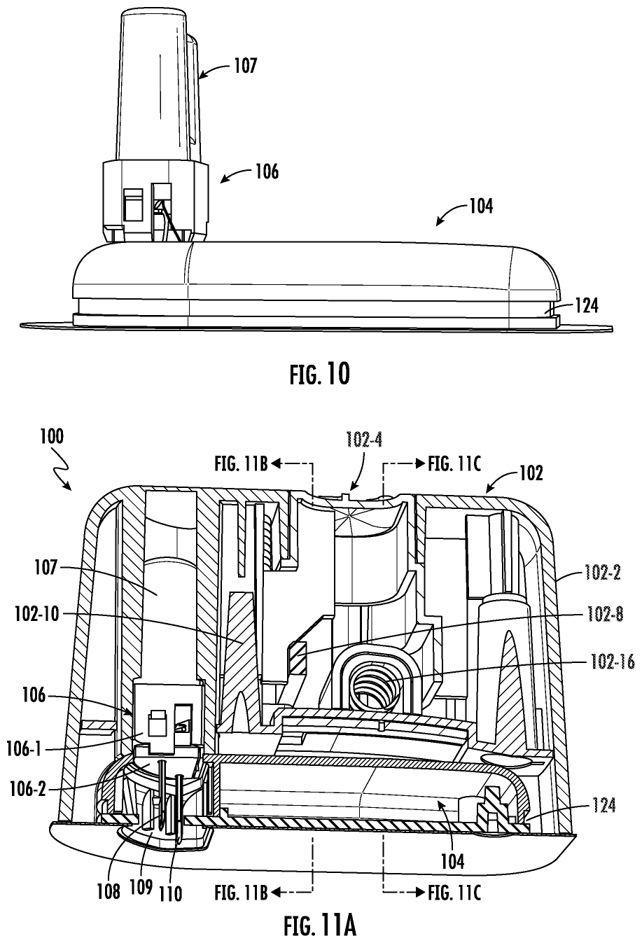

FIG. 10 depicts a view of cartridge assembly 106 installed within device 104 but before it is assembled into detachable activation mechanism 102.

FIGS. 11A-11C depicts various views of infusion system 100 in which device 104 for delivering insulin is in a pre-activation configuration that is assembled into detachable activation mechanism 102. Cartridge assembly 106 is in an upward position. The spring for introducer needle insertion and device 104 release are loaded as shown. In more detail, in this configuration, device 104 for delivering insulin is securely attached to and within the detachable activation mechanism 102 by way of retention flanges 102-12a, 102-14a on arms 102-12, 102-14), respectively. These flanges 102-12a, 102-14a are biased outwardly by release spring 102-16. In the locked position, retention flanges 102-12a, 102-14a engage groove 124 of device 104 to secure device 104 within detachable activation mechanism 102. Activation beam 102-8 holds insertion rod 102-22 in an upward position and spring 102-6 loaded. Button features or ledges 102-4b, 102-4c lock the release arms 102-12, 102-14 in the closed position so that flanges 102-12a, 102-14a within groove 124 lock the device 104 in detachable activation mechanism 102. Button ledges 102-4b, 102-4c interfere with the release arms (e.g., arm 102-12) before activation.

When activation button assembly 102-4 is pressed, the ramped surface 102-4a on button assembly 102-4 translates downward, causing the activation beam 102-8 to pivot, freeing the release of insertion rod 102-22. Under load from spring 102-6, insertion rod 102-22 moves downward, thereby forcing cartridge assembly 106 downward as well. Insertion rod 102-22 pushes both CGM needle 112 and infusion needle 114 into an inserted position within the subcutaneous tissue of the user. Simultaneously, when button assembly 102-4 translates downward by force from the user, button ledges 102-4b, 102-4c of button assembly 102-4 (of detachable activation mechanism 102) move into a position without interference from the release arms and spring 102-16 causes retention flanges 102-12a, 102-14a of the release arms 102-12,102-14 to move outwardly, thereby moving flanges out of a groove 124 (also called indentation), releasing device 104.

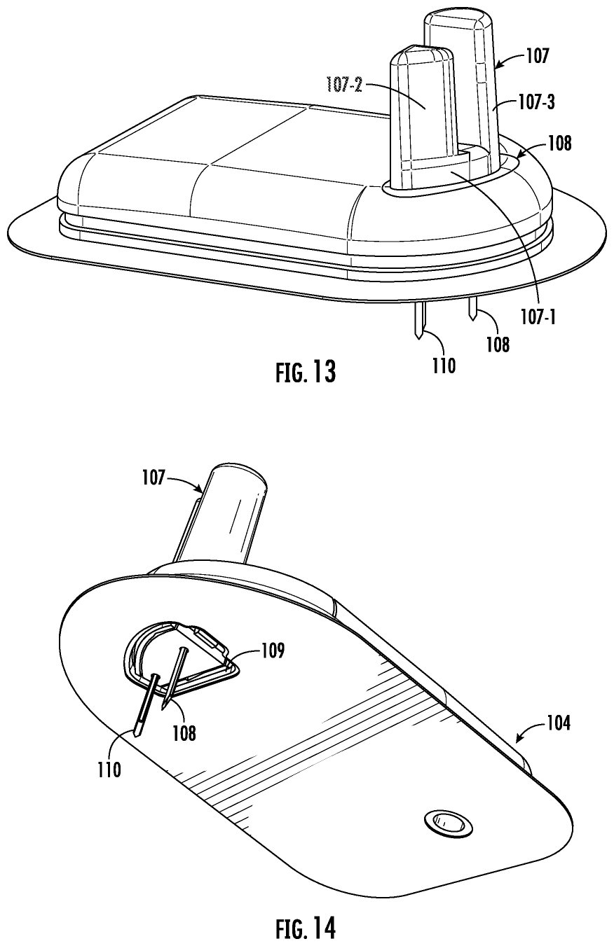

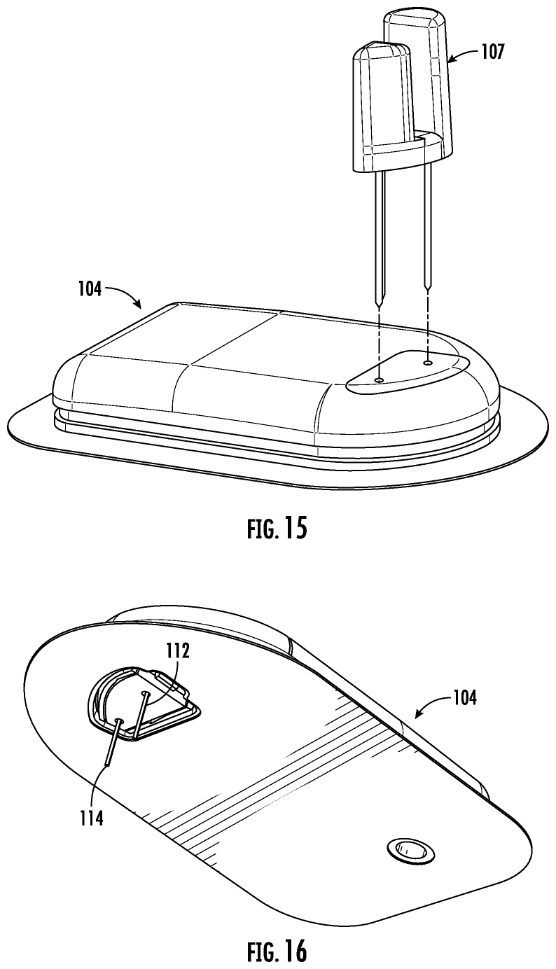

FIG. 12 depicts a transparent view of infusion system 100 in which button 102-4 has been pressed which rotates a release beam allowing an insertion rod to drive introducer needles 108,110 into a user's tissue. Introducer needles 108,110 remain in the tissue. After activation, the user removes detachable activation mechanism 102 leaving device 104 as shown in FIGS. 13 and 14. Insertion needle hub 107 protrudes from device 104 and is used as a handle to manually pull-out (withdraw) introducer needles 108,110. This is shown in FIG. 15. FIG. 16 depicts a view of device 14 with introducer needles 108, 110 removed. In structural detail, in this example, hub 107 includes base 107-1 and dual towers 107-2, 107-3. Dual towers 107-2, 107-3 are configured to enable a user to grasp and manually pull-out (withdraw) introducer needles 108,110. However, hub 107 may be configured in any size or shape to enable a user to easily withdraw introducer needles 108,110.

FIG. 17 depicts a block diagram of example components of (1) device 1700 for delivering insulin and (2) cartridge assembly 1702 of infusion system 100 as described in detail above. (Device 104 and cartridge assembly 106 are renumbered as device 1700 and cartridge assembly 1702 in FIG. 17.) Specifically, device 1700 incorporates several components or modules (not shown) in the fluidic pathway including reservoir 1700-1 for storing the insulin, micropump 1700-2 for pumping the insulin, sensors 1700-3 (e.g., pressure) for sensing various parameters in the system and user and tubing connecting infusion needle 1702-1 to reservoir 1700-1 within cartridge assembly 1702. Device 1700 also includes microcontroller unit (MCU) 1700-4 and battery and power controller 1700-5. Cartridge assembly 1702 also includes CGM sensor 1700-5. CGM, as known to those skilled in the art, tracks user glucose levels and permits those levels to be used in algorithms that control flow rate. MCU 1700-4 controls the operation of micropump 1700-2. Infusion catheter 1702-1 and CGM sensor 1702-2 are shown in FIG. 17. However, the hub and the introducer needles described hereinabove (for infusion catheter 1702-1 and CGM sensor 1702-2) are not shown in FIG. 17.

Reservoir 1700-1 is configured to receive and store insulin for its delivery over a course of about three days, or as needed. However, reservoir size may be configured for storing any quantity of fluid as required.

MCU 1700-5 electronically communicates with sensors 1700-3 and micropump 1700-2 as well as the CGM sensor 1702-2, as the monitoring components. Among several functions, MCU 1700-5 operates to control the operation of micropump 1700-2 to deliver insulin through insulin needle 1702-1 from reservoir 1700-1 at specific doses, i.e., flow rates over specified time intervals, based on CGM data converted to desired flow rate via control algorithms.

Battery and power controller 1700-4 controls the power to MCU 1700-5 and micropump 1700-2 to enable those components to function properly as known to those skilled in the art. CGM sensor 1700-2 is powered by battery and power controller 1700-4 through MCU 1700-5.

The components of device 1700 and cartridge assembly 1702 shown in FIG. 17 depict only a few components. Those skilled in the art know that device 1700 and cartridge assembly 1702 may include additional or less components as needed.

It is to be understood that the disclosure teaches examples of the illustrative embodiments and that many variations of the invention can easily be devised by those skilled in the art after reading this disclosure and that the scope of the present invention is to be determined by the claims below.

What is claimed is:

1. An infusion system comprising:
a device for delivering fluid to a tissue of the user;
a hub engaging the device for delivering fluid, the hub including an introducer needle for introducing an infusion catheter or an analyte sensor into the tissue of the user, the hub configured to be removable by the user; and
an activation mechanism for moving the hub toward the user to thereby cause the introducer needle to be inserted into the tissue of the user.

2. The infusion system of claim 1 further including a cartridge assembly engaging the hub, the cartridge assembly including the infusion catheter or the analyte sensor, the needle cartridge assembly configured to move in response to movement of the hub from (1) a telescoping position above the top surface of device, wherein the introducer needle is in a retracted position within the device for delivering insulin to the user and (2) an advanced position within the device, wherein the introducer needle is in a deployed position inserted into the tissue of the user along with the catheter or the analyte sensor.

3. The infusion system of claim 1 wherein the activation mechanism includes a spring and a button assembly, that upon activation by the user, releases the spring driving the hub toward the user and causes the introducer needle to be inserted into the tissue of the user.

4. The infusion system of claim 3 wherein the activation mechanism includes an insertion rod and a release beam configured to rotate from a first position wherein release beam supports the insertion rod to a second position, upon activation of the button assembly, wherein the release beam does not support the insertion rod, thereby releasing the spring.

5. The infusion system of claim 1 wherein the cartridge assembly includes a base insert and cartridge configured to secure the base insert therewithin.

6. The infusion system of claim 1 wherein the introducer needle is U-shaped.

7. The infusion system of claim 1 wherein the activation mechanism is a detachable activation mechanism.

8. The infusion system of claim 1 wherein the fluid is insulin.

9. The infusion system of claim 1 further including a cartridge assembly engaging the hub, the cartridge assembly including the infusion catheter or the analyte sensor, the needle cartridge assembly configured to move in response to movement of the hub from (1) a first position, wherein infusion catheter or analyte sensor are in a retracted position above the tissue of the user to (2) a second position, wherein the infusion catheter or analyte sensor are in a deployed position inserted into the tissue of the user.

10. An infusion system comprising:
a device for delivering fluid to a user, the device including a pump for pumping the fluid into a tissue of the user;
a cartridge assembly including an infusion catheter and an analyte sensor to be inserted into the tissue of the user, the needle cartridge assembly configured to move from (1) a first position, wherein infusion catheter and analyte sensor are in a retracted position above the tissue of the user to (2) a second position, wherein the infusion catheter and analyte sensor are in a deployed position inserted into the tissue of the user; and
a hub configured to engage the cartridge assembly and to be removed from the cartridge assembly, the hub including first and second introducer needles for introducing the infusion catheter and the analyte sensor, respectively into the tissue of the user.

11. The infusion system of claim 10 further comprising an activation mechanism for causing the cartridge assembly to move from (1) the telescoping position to (2) the advanced position within the device.

12. The infusion system of claim 11 wherein the activation mechanism includes a spring and a button assembly, that upon activation by the user, releases the spring driving the hub toward the user and causes the introducer needle to be inserted into the tissue of the user.

13. The infusion system of claim 12 wherein the activation mechanism further includes an insertion rod and a release beam configured to rotate from a first position wherein the release beam supports the insertion rod to a second position, upon activation of the button assembly, wherein the release beam does not support the insertion rod, thereby releasing the spring.

14. The infusion system of claim 10 wherein the first and/or second introducer needles are U-shaped.

15. The infusion system of claim 10 wherein the activation mechanism is a detachable activation mechanism.

16. The infusion system of claim 10 wherein the cartridge assembly includes a base insert and cartridge configured to secure the base insert therewithin.

17. An infusion system comprising:
a device for delivering insulin to a user, the device including a pump for pumping the insulin into a subcutaneous tissue layer of the user;
a cartridge assembly including an infusion catheter and an analyte sensor to be inserted into the subcutaneous tissue layer of the user, the needle cartridge assembly configured to move from (1) a telescoping position above the top surface of device, wherein the introducer needle is in a retracted position within the device for delivering insulin to the user and (2) an advanced position within the device, wherein the introducer needle is in a deployed position inserted into the subcutaneous tissue layer of the user along with the catheter and the analyte sensor;
a hub configured to be removed from the cartridge assembly, the hub including first and second introducer needles for introducing the infusion catheter and the analyte sensor, respectively into the subcutaneous tissue layer of the user; and
a detachable activation mechanism for causing the cartridge assembly to move from (1) the telescoping position to (2) the advanced position within the device.

18. The infusion system of claim 17 wherein the activation mechanism includes a spring and a button assembly, that upon activation by the user, releases the spring driving the hub toward the user and causes the introducer needle to be inserted into the subcutaneous tissue layer of the user.

19. The infusion system of claim 18 wherein the activation mechanism further includes an insertion rod and a release beam configured to rotate from a first position wherein release beam supports the insertion rod to a second position, upon activation of the button assembly, wherein the release beam does not support the insertion rod, thereby releasing the spring.

20. The infusion system of claim 17 wherein the introducer needle is U-shaped.
